(19) United States
(12) Patent Application Publication
(10) Pub. No.: US 2023/0329533 A1
(43) Pub. Date: Oct. 19, 2023

(54) ILLUMINATION DEVICE FOR A SURGICAL INSTRUMENT, SURGICAL SYSTEM, METHOD OF OPERATING AN ILLUMINATION DEVICE AND A SURGICAL SYSTEM

(71) Applicant: OLYMPUS Winter & Ibe GmbH, Hamburg (DE)

(72) Inventor: Konrad ANDERS, Hamburg (DE)

(73) Assignee: OLYMPUS Winter & Ibe GmbH, Hamburg (DE)

(21) Appl. No.: 18/125,226

(22) Filed: Mar. 23, 2023

Related U.S. Application Data
(60) Provisional application No. 63/332,354, filed on Apr. 19, 2022.

(51) Int. Cl. A61B 1/00; F21V 8/00; G02B 6/04

(52) U.S. Cl. CPC A61B 1/00167; A61B 1/00006; G02B 6/0006; G02B 6/04

(57) ABSTRACT

An illumination device including an illumination source having a light source; a sensor; and a processor; and a light guiding cable having main and secondary fiber bundles. The light guiding cable comprises a first end coupled to the illumination source and an opposite second end configured for coupling with a surgical instrument such that the light source provides the surgical instrument with light from the light source. The first end is coupled to the illumination source such that light from the light source is coupled into the main fiber bundle and light guided in the secondary fiber bundle is directed to the sensor. The processor receives a signal from the sensor indicative of an illumination level at the sensor and generates a control signal if the illumination level of the sensor is above a predetermined threshold value.

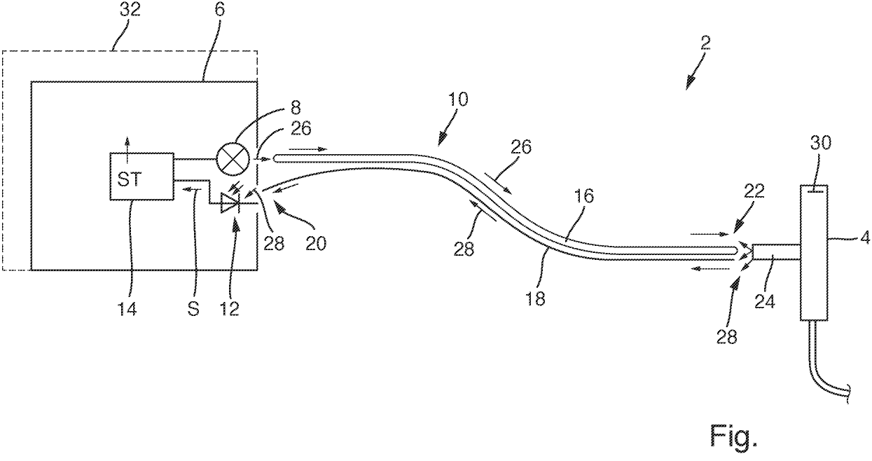

### CROSS-REFERENCE TO RELATED APPLICATION

The present application is based upon and claims the benefit of priority from U.S. Provisional Application No. 63/332,354 filed on Apr. 19, 2022, the entire contents of which is incorporated herein by reference.

### BACKGROUND

### Field

The present disclosure relates to an illumination device for a surgical instrument, and more particularly to an illumination device for a surgical instrument comprising an illumination source and a light guiding cable. Further, the present disclosure relates to a surgical system, and more particularly to a surgical system comprising such a surgical instrument. The present disclosure also relates to a method of operating an illumination device for a surgical instrument and to a method of operating a surgical system.

### Prior Art

Medical procedures often require an illumination source to allow the medical professional to visualize the surgical side. This also applies to endoscopes, which illuminate an area of interest near their distal end. Because light sources inevitably generate lost heat, and uncontrolled heating of a surgical site is undesirable, the light source is frequently arranged distanced from the endoscope or surgical instrument. To transport light from an illumination source, for example an LED, of an illumination device to the endoscope, a light guiding cable is used. A first end of this light guiding cable is coupled to the illumination device with its entrance side and an opposite second end is connected to the endoscope, with the exit side of the light guiding cable. A light guiding cable typically comprises of a fiber bundle of many single glass fibers and is covered by a plastic or a rubber cladding to protect the fiber bundle. An endoscope, which is connected to an illumination device is for example known from US 2019/0038120 A1.

The exit port of the illumination device, to which the light guiding cable is connected, is typically provided with an electrical or mechanical shutter to prevent light, which is generated by an illumination source inside the illumination device from exiting the device in case no light guiding cable is connected to the exit port. The free and second end of the light guiding cable, which provides the interface to the surgical instrument, does however not comprise such a shutter device. Consequently, in a situation in which the entrance side of the light guiding cable is connected to the illumination device, but the exit end of the light guiding cable is not connected to the surgical instrument, there is a risk of light exiting the light guiding cable at this free end, when the illumination device is accidentally operated.

This can expose the medical staff to potential risks, such as if the illumination device comprises a laser as the illumination source. To avoid such a situation, light guiding cables are configured with an additional electric cable and a suitable sensor configured to detect, whether or not the light guiding cable is appropriately connected to the surgical instrument. The illumination source is configured in that it can only be switched to a mode of operation, in which the illumination source emits light radiation, when a signal of the sensor is communicated back to the illumination device, which is indicative of the appropriate connection of the light guiding cable to the surgical instrument. This ensures that the illumination device is not operated in that, for example a laser radiation can be emitted from the free and second end of the light guiding cable.

This type of safety circuit, however, requires the integration of an electrical cable in the light guiding cable. Furthermore, an appropriate sensor and corresponding electronics have to be placed at the second and free end of the light guiding cable and in the illumination device.

### SUMMARY

An object is to provide an illumination device for a surgical instrument and a surgical system having a simplified configuration and to provide a simplified method of operating an illumination device and a simplified method of operating the surgical system.

Such object can be solved by an illumination device for a surgical instrument, comprising an illumination source and a light guiding cable, wherein
the illumination source comprises at least one light source, a sensor and a processor comprsing hardware, and the light guiding cable comprises a main fiber bundle and a secondary fiber bundle,
the light guiding cable comprises a first end and an opposite second end, the first end being coupled to the illumination source and the second end being configured for coupling with the surgical instrument, wherein the light guiding cable is configured to couple the illumination device and the surgical instrument in that the light source can provide the surgical instrument with light from the light source,
the first end of the light guiding cable is coupled to the illumination source in that light from the light source is coupled into the main fiber bundle of the light guiding cable and light guided in the secondary fiber bundle is detected by the sensor,
the processoris coupled to the sensor and is configured to receive a signal from the sensor indicative of an illumination level at the sensor, wherein the processoris further configured to issue a signal, if the illumination level of the sensor is above a predetermined threshold value.

Thus, at the connecting port of the surgical instrument at which the light guiding cable is connected thereto, a certain amount of stray light will occur. The stray light results from inevitable reflections that occur when coupling a light from the second end of the light guiding cable into the surgical instrument. These reflections are minimal and do not in fact impair the optical coupling between the light guiding cable and the surgical instrument. It is typically not possible and also not desirable to completely avoid these reflections.

The connecting port of the surgical instrument, at which the light guiding cable is connected thereto can be configured in that a predetermined amount of stray light is generated. This can be for example performed by a suitable configuration of surfaces, at which reflections occur. For example a coating at these surfaces can be configured so as to generate a certain amount of reflection which in turn results in a certain amount of stray light. The specific generation of stray light at the connecting port simplifies the detection of the coupling between the light guiding cable and the surgical instrument.

The stray light couples into the secondary fiber bundle of the light guiding cable at said second end. The stray light is guided in the secondary fiber bundle and can be detected by the sensor. Hence, the signal which is detected by the sensor indicates whether or not an endoscope or another surgical instrument is coupled to the second end of the light guiding cable.

When the illumination source of the illumination device is switched on, a minimum of stray light, which can be due to reflections between the connecting port of the surgical instrument and the second end of the light guiding cable, will be generated. As it is outlined above, this stray light will couple into the secondary fiber bundle of the light guiding cable and can be detected on the sensor in the illumination device. Only if this signal exceeds a predetermined threshold value, which means that the surgical instrument is coupled to the second end of the light guiding cable, a primary light source of the illumination device can be switched on. This applies to situations, in which the illumination device comprises a primary and a secondary light source, for example a laser as the primary light source and an LED or other less intense light source as the secondary light source. This secondary and less intense light source can be applied for determination of the connecting status of the illumination device, as will be outlined in the following in more detail. Thus, the primary light source can only switched on when appropriate connection of the light guiding cable has been tested using the secondary light source. The approval for safe operation of the secondary light source can be triggered by the signal, which is indicative of the illumination level of the sensor and which is issued by the processor inside the illumination device.

Work safety can be enhanced. There is no risk for uncontrolled light emission at the second and free end of the light guiding cable by the illumination source if no surgical instrument is connected, including where the at least one light source is a laser radiation source or comprises a laser radiation source. Naturally, this also applies to other high-intensity light sources.

The light guiding cable can this be configured to only comprise fiber bundles, namely the main fiber bundle and the secondary fiber bundle, and no electrical wire. It is therefore not necessary to combine production technologies from different technical fields with each other, which is the case when a light guiding cable comprises not only light guiding glass fibers but also electrical wires. The light guiding cable can be manufactured using only optical components, i.e. optical fibers. Furthermore, it is not necessary to include any sensor device at the free and second end of the light guiding cable. In contrast to this, the sensors and the processor can be arranged in the illumination device itself.

The processor can be configured to operate the light source and the sensor in a test mode in that the light source emits light into the main fiber bundle and an illumination level is detected at the sensor at the same time.

In the test mode, the light source or one of the light sources of the illumination device emits light. Stray light, which is due to this light emission, is detected at the sensor. Therefore, a correct connection of the surgical instrument to the free end second end of the light guiding cable can be detected.

The illumination source can comprise a first light source and a second light source, wherein the first light source has a higher intensity than the second light source. The illumination source can comprise a laser, such as a semiconductor laser, as the first light source and a white light source as the second light source. The second light source can be operated in the test mode and the processor can be further configured to enable operation of the first light source only upon receipt of said signal.

The illumination device can be operated with enhanced work safety even where the first light source is a laser light source.

According to still another embodiment, the processor can be configured to check presence of the control signal and to continue operation of the laser only when the presence of the control signal is confirmed.

The illumination device can check the presence of stray light to continue operation of the laser. The presence of stray light indicates that the second end of the light guiding cable is connected to the surgical instrument. In this way, it is ensured that no laser light will emit from the second end of the light guiding cable if the same is not properly connected. Thus, work safety can be further enhanced.

In still another embodiment, the processor can be further configured to detect an illumination level of laser light at the sensor and to receive a signal from the sensor, which is indicative of a laser light illumination level at the sensor, wherein the processor is configured to issue a further control signal, if the laser light illumination level at the sensor is above a predetermined laser-light-threshold value.

The further control signal can replace the control signal which is mentioned above. In other words, operation of the laser can be continued if laser stray light is detected. This can further enhance the work safety, because appropriate connection of the second end of the light guiding cable is detected by direct detection of laser stray light. For detection of the laser stray light, the above referred detector can be applied. According to a further embodiment, the illumination device can comprise a separate and further sensor, which is configured for detection of the laser straylight. This detector can be equipped with an appropriate filter, which can be configured in that only the laser wavelength is transmitted by said filter. By this measure exclusive detection of laser stray light can be performed.

According to another embodiment, the main fiber bundle and the secondary fiber bundle can each comprise a plurality of optical fibers, wherein a number of optical fibers in the main fiber bundle can exceed a number of optical fibers in the secondary fiber bundle.

The configuration of the light guiding cable, in which the secondary fiber bundle can have a lower number of optical fibers in comparison to the main fiber bundle, can result in an economic configuration of the light guiding cable. According to still another embodiment, a diameter of the secondary fiber bundle can be smaller than a diameter of the main fiber bundle.

Such object can be further solved by a surgical system comprising a surgical instrument, such as a surgical endoscope, and an illumination device according to one or more of the preceding embodiments, wherein the second end of the light guiding cable of the illumination device is coupled to the surgical instrument at a light connecting port of the surgical instrument.

Same or similar features and/or results, which have been mentioned with respect to the illumination device also apply to the surgical system and should therefore not be repeated.

The second end of the light guiding cable can be coupled to the light connecting port in that light emitted from the main fiber bundle at the second end of the light guiding cable can generate stray light, which is coupled into the secondary fiber bundle at the second end of the light guiding cable.

The surgical instrument can comprise an image sensor and the surgical system can further comprise a video device configured to process video data received from the image sensor, and wherein the illumination device is a component of the video device.

Such object can be further solved by a method of operating an illumination device for a surgical instrument, the illumination device comprising an illumination source and a light guiding cable, wherein
the illumination source comprises at least one light source, a sensor and a processor comprising hardware and the light guiding cable comprises a main fiber bundle and a secondary fiber bundle,
the light guiding cable comprises a first end and an opposite second end, the first end being coupled to the illumination source and the second end being configured for coupling with the surgical instrument, wherein the light guiding cable is for coupling the illumination device and the surgical instrument in that the light source provides the surgical instrument with light from the light source,
the first end of the light guiding cable is coupled to the illumination source in that light from the light source is coupled into the main fiber bundle of the light guiding cable and light guided in the secondary fiber bundle is detected by the sensor,
the processor is coupled to the sensor and receives a signal from the sensor indicative of an illumination level at the sensor, wherein the processor issues a control signal, if the illumination level at the sensor is above a predetermined threshold value.

Also with respect to the method of operating an illumination device, same or similar features and/or results, which have been mentioned with respect to the illumination device, apply.

Furthermore, the processing device can operate the light source and the sensor in a test mode in that the light source emits light into the main fiber bundle and an illumination level is detected at the sensor at the same time.

The illumination source can comprise a laser, such as a semiconductor laser, as a first light source and a white light source as a second light source, wherein the second light source can be operated in the test mode and the processor can enable operation of the laser only upon presence of the control signal.

The processor can check presence of the control signal and continue operation of the laser only when the presence of the control signal is confirmed.

The processor can detect an illumination level of laser light at the sensor and receives a signal from the sensor, which is indicative of a laser light illumination level at the sensor, wherein the processor can issue a further control signal, if the laser light illumination level at the sensor is above a predetermined laser-light-threshold value.

Furthermore, such object can be solved by a method of operating a surgical system comprising a surgical instrument, such as a surgical endoscope, and an illumination device comprising an illumination source and a light guiding cable, wherein
the illumination source comprises at least one light source, a sensor and a processor comprising hardware and the light guiding cable comprises a main fiber bundle and a secondary fiber bundle,
the light guiding cable comprises a first end and an opposite second end, the first end being coupled to the illumination source and the second end being configured for coupling with the surgical instrument, wherein the light guiding cable is for coupling the illumination device and the surgical instrument in that the light source provides the surgical instrument with light from the light source,
the first end of the light guiding cable is coupled to the illumination source in that light from the light source is coupled into the main fiber bundle of the light guiding cable and light guided in the secondary fiber bundle is detected by the sensor,
the processor is coupled to the sensor and receives a signal from the sensor indicative of an illumination level at the sensor, wherein the processor issues a control signal, if the illumination level of the sensor is above a predetermined threshold value, wherein
the second end of the light guiding cable of the illumination device is coupled to the surgical instrument at a light connecting port of the surgical instrument and the second end of the light guiding cable is coupled to the light connecting port in that light emitted from the main fiber bundle at the second end of the light guiding cable generates stray light, which is coupled into the secondary fiber bundle at the second end of the light guiding cable, wherein
the processor operates the light source and the sensor in a test mode in that the light source emits light into the main fiber bundle and an illumination level is detected at the sensor at the same time, wherein
the illumination source comprises a laser, such as a semiconductor laser, as a first light source and a white light source as a second light source, and wherein the second light source is operated in the test mode and the processor enables operation of the laser only upon presence of the control signal.

Same or similar features and/or results, which have been mentioned with respect to the illumination device also apply to the method of operating the illumination device and the method of operating the surgical system in the same or a similar way and are therefore not repeated.

### BRIEF DESCRIPTION OF THE DRAWINGS

Further characteristics will become apparent from the description of the embodiments together with the claims and the included drawing. Embodiments can fulfill individual characteristics or a combination of several characteristics.

The embodiments are described below, without restricting the general intent of the invention, based on the exemplary embodiments, wherein reference is made expressly to the drawing with regard to the disclosure of all details that are not explained in greater detail in the text. In the drawings:

the FIGURE. illustrates a simplified schematic illustration of a surgical system comprising an illumination device and a surgical instrument, which is an endoscope by way of an example.

In the drawing, the same or similar types of elements or respectively corresponding parts are provided with the same reference numbers in order to prevent the item from needing to be reintroduced.

### DETAILED DESCRIPTION

In the FIGURE., there is a surgical system 2 comprising a surgical instrument 4, which is by way of an example only, a surgical endoscope. Furthermore, the surgical system 2 comprises an illumination device 6. The illumination device 6 comprises an illumination source 8, a light guiding cable 10, a sensor 12 and a processing device (processor comprising hardware) 14, such as one or more circuits, a CPU, controller, computer and the like comprising hardware, which includes a processor configured from hardware or a combination of hardware and software. The illumination source 8 comprises at least one light source, for example an LED or a laser. The illumination source 8 can comprise a first and a second light source to which reference will be made further below.

The light guiding cable 10 comprises a main fiber bundle 16 and a secondary fiber bundle 18. The two fiber bundles 16, 18 are covered by a common cladding, which is for example made of a synthetic material or from rubber and which is not shown in the FIGURE.

The light guiding cable 10 comprises a first end 20 and an opposite second end 22. The first end 20 is coupled to the illumination source 8 of the illumination device 6 by a first end connector. The second end 22 is configured for coupling with the surgical instrument 4 by a second end connector. The light guiding cable 10 is configured to couple the illumination device 6 and the surgical instrument 4 in that light from the illumination source 8 can be provided to the surgical instrument 4. For that purpose, the light guiding cable 10 can be coupled or fixed to the illumination device 6 using a suitable first end connector. At the second end 22, the light guiding cable 10 can be provided with a further suitable second end connector, which fits into a connecting port 24 of the surgical instrument 4. The first end 20 of the light guiding cable 10 is coupled to the illumination source 8 in that light from the light source, for example the LED or laser, is coupled into the main fiber bundle 16 of the light guiding cable 10. Furthermore, the first end 20 of the light guiding cable 10 is coupled to the illumination device 6 in that light, which is guided in the secondary fiber bundle 18, is directed onto the sensor 12.

In a situation, in which the illumination source 8 emits light 26, which is coupled into the main fiber 16 and guided in the main fiber 16, there is a stray light 28 occurring at the connection port 24 of the surgical instrument 4. The stray light 28 results from inevitable reflections at the port 24 and the second end 22 of the light guiding cable 10. The intensity of the stray light 28 is very low when compared to the overall intensity of light 26, which is coupled into the port 24 of the surgical instrument 4. It does not impair the optical coupling between the light guiding cable 10 and the surgical instrument 4. It is, however, typically not possible and also satisfies no technical goal to completely avoid reflections and the occurrence of a minimum level of stray light 28. The second end 22 of the light guiding cable 10 is coupled to the port 24 of the surgical instrument 4 in that stray light 28 occurring at the port 24 is coupled into the secondary fiber bundle 18. The stray light 28 is guided in the secondary fiber bundle 18 and is directed onto the sensor 12 in the illumination device 6.

The processing device 14 of the illumination device 6 is coupled to the sensor 12 and is configured to receive a signal S from the sensor 12, which is indicative of an intensity of illumination, i.e., an illumination level at the sensor 12. The processing device 14 is further configured to issue a control signal ST if the illumination level at the sensor 12 exceeds a predetermined threshold value. The aforementioned signal S is a measure of the illumination level at the sensor 12. If the illumination level is above a certain threshold value, it can be assumed that the second end 22 of the light guiding cable 10 is coupled to the port 24. Hence, by analyzing the signal S, appropriate connection of the light guiding cable to the port 24 can be determined. The control signal ST indicates that the connection is appropriate.

The processing device 14 is further configured to operate the illumination source 8 and the sensor 12 in a test mode. In said test mode, the illumination source 8 emits a light 26, which is coupled into the main fiber bundle. An illumination level is detected at the sensor 12 at the same time. By operating the illumination device 6 in the test mode, appropriate connection of the second end 22 of the light guiding cable 10 to the surgical instrument 4 can be tested.

Furthermore, the illumination device 6 can comprise a first light source and a second light source, which together form the illumination source 8. The second light source has a lower intensity, when compared to the first light source. For example, the first light source is a laser, such as a semiconductor laser, and the second light source is a white light source. The second light source is operated in the above-referred test mode and only if the signal S of the sensor 12 exceeds the predetermined threshold value and appropriate connection of the second end 22 of the light guiding cable 10 is detected, the first light source, for example the laser, can be switched on. This avoids uncontrolled emission of laser light at the second end 22 of the light guiding cable 10.

The surgical system 2, which is depicted in the FIGURE, can comprise a surgical instrument 4 having an image sensor 30 and the illumination device 6 forms part of a video device 32, which is configured to process video data from the image sensor 30.

While there has been shown and described what is considered to be embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

List of References

| | |
|---|---|
| 2 | Surgical system |
| 4 | Surgical instrument |
| 6 | Illumination device |
| 8 | Illumination source |
| 10 | Light guiding cable |
| 12 | Sensor |
| 14 | Processing device |
| 16 | Main fiber bundle |
| 18 | Secondary fiber bundle |
| 20 | First end |
| 22 | Second end |
| 24 | Port |
| 26 | Light |
| 28 | Stray light |
| 30 | Image sensor |
| 32 | Video device |
| S | Signal |
| ST | Control signal |

What is claimed is:

1. An illumination device comprising:
an illumination source comprising:
at least one light source;
a sensor; and
a processor comprising hardware; and
a light guiding cable comprising:
a main fiber bundle; and
a secondary fiber bundle;
wherein the light guiding cable comprises a first end and an opposite second end, the first end being coupled to the illumination source and the second end being configured for coupling with a surgical instrument to couple the illumination device and the surgical instrument such that the light source provides the surgical instrument with light from the light source,
the first end of the light guiding cable is coupled to the illumination source such that light from the light source is coupled into the main fiber bundle of the light guiding cable and light guided in the secondary fiber bundle is directed to the sensor, and
the processor is configured to:
receive a signal from the sensor indicative of an illumination level at the sensor; and
generate a control signal, if the illumination level of the sensor is above a predetermined threshold value.

2. The illumination device according to claim 1, wherein the processor is configured to operate the illumination source and the sensor in a test mode such that the processor controls the illumination source to emit test light into the main fiber bundle and detects the illumination level at the sensor based on the test light.

3. The illumination device according to claim 2, wherein the at least one light source comprises a first light source and a second light source, the first light source comprising a laser and the second light source comprising a white light source, wherein the second light source is operated in the test mode and the processor is further configured to enable operation of the laser only where the control signal is generated.

4. The illumination device according to claim 3, wherein the processor is further configured to continue operation of the laser only when the control signal is not generated.

5. The illumination device according to claim 3, wherein the processor is further configured to:
detect an illumination level of laser light at the sensor to receive the signal from the sensor, which is indicative of the light illumination level from the laser light at the sensor, and
generating the control signal if the illumination level at the sensor from the laser light is above the predetermined threshold value.

6. The illumination device according to claim 1, wherein the main fiber bundle comprises a first plurality of optical fibers and the secondary fiber bundle comprises a second plurality of optical fibers, wherein a first number of the first plurality of optical fibers exceeds a second number of the second plurality of optical fibers.

7. A surgical system comprising:
the illumination device according to claim 1; and
the surgical instrument having a light connecting port;
wherein the second end of the light guiding cable is coupled to the light connecting port of the surgical instrument.

8. The surgical system according to claim 7, wherein the second end of the light guiding cable is coupled to the light connecting port in that light emitted from the main fiber bundle at the second end of the light guiding cable generates stray light, which is coupled into the secondary fiber bundle at the second end of the light guiding cable.

9. The surgical system according to claim 7, wherein the surgical instrument comprises an image sensor and the surgical system further comprises a video device configured to process video data received from the image sensor, and the illumination device is a component of the video device.

10. A method of operating an illumination device comprising a light source, a sensor and a light guiding cable, the method comprising:
coupling a first end of the light guiding cable to the light source such that light from the light source is coupled into a main fiber bundle of the light guiding cable and light guided in a secondary fiber bundle of the light guiding cable is detected by the sensor;
coupling a second end of the light guiding cable to the surgical instrument to provide the surgical instrument with light from the light source,
receiving a signal from the sensor indicative of an illumination level at the sensor; and
generating a control signal if the illumination level at the sensor is above a predetermined threshold value.

11. The method according to claim 10, wherein the further comprising operating the light source and the sensor in a test mode in that the light source emits test light into the main fiber bundle and the illumination level is detected at the sensor based on the test light.

12. The method according to claim 11, wherein the light source comprises a first light source and a second light source, the first light source comprising a laser and the second light source comprising a white light source, wherein the test light comprises light from the second light source and the laser is enabled only where the control signal is generated.

13. The method according to claim 12, further comprising continuing operation of the laser only where the control signal is not generated.

14. The method according to claim 12, further comprising:
detecting an illumination level of laser light at the sensor to receive the signal from the sensor, which is indicative of the light illumination level from the laser light at the sensor, and
generating the control signal if the illumination level at the sensor from the laser light is above the predetermined threshold value.

15. An apparatus for operating an illumination device comprising a light source, a sensor and a light guiding cable, the apparatus comprising:
a processor comprising hardware, the processor being configured to:
operate the light source to emit illumination light into a first portion of a light bundle disposed in the light guiding cable connected to a surgical instrument;
receive a signal from a sensor indicative of an amount of stray illumination level at the sensor, the stray illumination coming from the surgical instrument by a second portion of the light bundle disposed in the light guiding cable; and
generate a control signal if the stray illumination level at the sensor is above a predetermined threshold value.

16. The apparatus according to claim 15, wherein the processor is further configured to operate the light source and the sensor in a test mode in that the light source emits test light into the first portion of the light bundle and the illumination level is detected at the sensor based on the test light.

17. The apparatus according to claim 16, wherein the light source comprises a first light source and a second light source, the first light source comprising a laser and the second light source comprising a white light source, wherein processor is configured to operate the second light source in the test mode and the processor is further configured to operate the laser only where the control signal is generated.

18. The apparatus according to claim 17, wherein the processor is further configured to continue operation of the laser only where the control signal is not generated.

19. The apparatus according to claim 17, wherein the processor is configured to:
detect an illumination level of laser light at the sensor to receive the signal from the sensor, which is indicative of the light illumination level from the laser light at the sensor, and
generate the control signal if the illumination level at the sensor from the laser light is above the predetermined threshold value.
